(19) United States
(12) Patent Application Publication
(10) Pub. No.: US 2026/0234691 A1
(43) Pub. Date: Aug. 13, 2026

(54) Point-of-Care Device for the Determination of Adenosine Deaminase Activity in Biological Samples

(71) Applicants: IN VITRO DIAGNOSTIC SOLUTIONS, Mount Laurel, NJ (US); DADA2 Foundation, Nashville, TN (US)

(72) Inventors: Robert Harper, Mount Laurel, NJ (US); Lex M. Cowsert, Nashville, TN (US); Eugene Chambers, Nashville, TN (US)

(21) Appl. No.: 19/154,578

(22) PCT Filed: Feb. 9, 2024
(86) PCT No.: PCT/US2024/015155

§ 371 Date: Aug. 7, 2025

Related U.S. Application Data
(60) Provisional application No. 63/484,279, filed on Feb. 10, 2023.

(51) Int. Cl. C12Q 1/34; C12Q 1/28; G01N 21/77; G01N 21/78; G01N 21/84

(52) U.S. Cl. CPC C12Q 1/34; C12Q 1/28; G01N 21/78; G01N 21/8483; G01N 2021/7759; G01N 2201/0221; G01N 2333/9029; G01N 2333/908; G01N 2333/978; G01N 2800/04

(57) ABSTRACT

Devices, kits and methods for testing and monitoring total adenosine deaminase and adenosine deaminase-2 (ADA2) activities and calculating adenosine deaminase-1 (ADA1) activity in biological samples are provided.

This patent application claims the benefit of priority from U.S. Provisional Application Ser. No. 63/484,279 filed Feb. 10, 2023, teachings of which are herein incorporated by reference in their entirety.

### FIELD OF THE DISCLOSURE

The disclosure relates to devices and methods for use of the devices for testing and monitoring the activity of total adenosine deaminase (ADA) activity and adenosine deaminase isoforms ADA1 and ADA2 in biological samples. In one non-limiting embodiment, the devices are used at the point-of-care.

### BACKGROUND

Adenosine deaminase (ADA) is an enzyme within the purine salvage pathway with a primary function of breaking down adenosine. Variants have been identified in bacteria, plants, invertebrates, vertebrates, and mammals with a highly conserved amino acid sequence suggesting ADA plays a crucial role in living tissue. It is expressed ubiquitously in the human body and plays an essential role in the development and maintenance of the immune system. Abnormal ADA expression or activity causes immune dysregulation and is associated with a significant amount of acute and chronic conditions.

Two isoforms of ADA have been identified in human tissue. ADA1 is found primarily in the cytosol, with the highest concentration observed in lymphocytes and macrophages, and ADA2 is found primarily in plasma and serum. The pathologies associated with either isoform are distinct.

Deficiency of ADA1 is a major cause of severe combined immunodeficiency (SCID) (Bradford et al. J. Clin. Immunol. 2017 37:626-637) and deficiency of ADA2 (DADA2) results in highly variable phenotypes including systemic vasculitis, early-onset stroke, bone marrow failure, and immunodeficiency (Zhou et al. N. Engl. J. Med. 2014 370:911-920; Navon Elkan et al. N. Engl. J. Med. 2014 370, 921-931). Early-onset cases of ADA deficiency are common and are generally more severe than late-onset cases.

Elevated ADA is also an important biomarker with diagnostic utility in tuberculosis (Lamsal et al. Southeast Asian J Trop Med Public Health 2007 38 (2):363-9), celiac disease (Cakal et al. Journal of Clinical Laboratory Analysis 2010 24:323-326), juvenile rheumatoid arthritis and systemic lupus erythematosus (Hitoglou et al. Clinical Rheumatology 2014 20:411-416; Saghiri et al. Rheumatoid Int 2012 32 (6):1633-8), and diabetes mellitus (Hoshino et al. Diabetes Res Clin Pract 1994 25 (2):97-102).

Presently there are limited treatments for many pathologies associated with abnormal ADA1 and ADA2.

ADA disorders are inherited in an autosomal recessive pattern and both ADA deficient individuals and carriers can be identified via genetic testing. Furthermore, biological ADA activity can be assessed via spectroscopic assays, enzyme-linked immunosorbent assay (ELISA), high-performance liquid chromatography (HPLC), or tandem mass spectrometry (MS/MS). Current methods require a centralized laboratory, expensive equipment, and trained personnel thus limiting their effectiveness. A rapid test for ADA activity, ideally at the point-of-care (POC), would have a great impact on reducing mortality and morbidity associated with this condition.

### SUMMARY

This disclosure relates to an electrochemical or colorimetric device with test strips, referred to herein as “ADA Now”, and methods and kits for use of this device with test strips for the quantitative determination of total ADA, ADA1 and/or ADA2 activity in biological samples. The device comprises a combination of components designed to elicit a measurable electrochemical or colored end-product from the application of a biological sample containing ADA. The device and method require less than 25 μL of a biological sample such as blood, plasma, serum, saliva, or urine for ADA quantitation. The end-electrical response or color of the reagent layer is indicative of total ADA or ADA2 activity and is proportional to the concentration of total ADA or ADA2 in the biological sample. Strategic segregation of a selective ADA1 inhibitor in one non-limiting test strip embodiment allows for simultaneous quantitation of total ADA and ADA2 activity with a calculated determination of ADA1 activity. The test strips, device, kits, and methods thereof provide quantitative results and are faster, more rugged, and easier to perform than analogous wet chemistry assays, lateral flow assays, or dedicated laboratory assays. The test strips, device, kits, and methods of this disclosure can be used at the point-of-care, at home, in the hospital, or at a clinician's office to measure total ADA and/or ADA2 and to calculate levels of ADA1, ADA2 and/or total ADA in biological samples.

### BRIEF DESCRIPTION OF DRAWINGS

FIG. 1 is a diagram of a non-limiting embodiment of a test strip of the present invention with the top and bottom of a cassette.

FIG. 2 is a photograph showing a non-limiting embodiment of a kit inclusive of a non-limiting embodiment of a test strip of this disclosure capable of measuring both total ADA and ADA2, means for obtaining a blood sample for testing, a portable hand-held meter for the test strips, and a mobile application for quantitative analysis and/or transmitting data from the test strip to a health care provider.

FIG. 3 is a graph showing the linear regression of calculated ADA2 levels in spiked plasma measured using a device in this disclosure versus levels confirmed by a reference assay (Diazyme Laboratories Inc., Poway, CA).

### DETAILED DESCRIPTION OF THE INVENTION

The present invention relates to a device and unique test strips for the determination of total ADA and/or ADA2 activity in biological samples as well as kits and methods for use of the test strips and device to measure total ADA and ADA2 activity and calculate ADA1 activity in biological samples. The test strips, devices, kits, and methods disclosed herein provide for a rapid, non-invasive, point-of-care test (POCT) for total ADA, ADA2, and calculated ADA1 activity.

The test strip of the present invention is useful in quantifying total ADA and ADA2 in biological samples of a subject and for detecting changes in total ADA and ADA2 levels over time in biological samples of a subject. In its simplest form, the test strip is comprised of a means for evenly spreading the applied sample, separating blood components from plasma, and generating an electrochemical or colorimetric response proportional to total ADA and/or ADA2 activity.

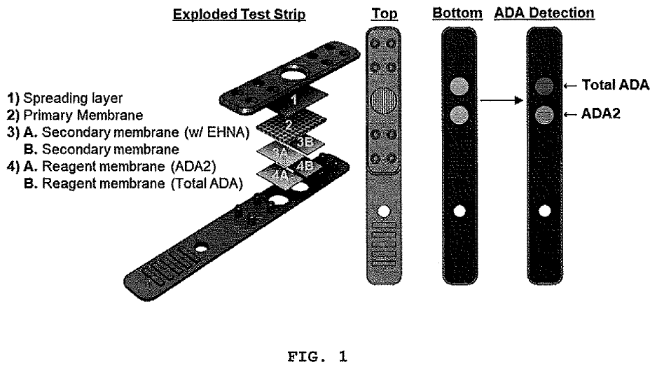

In one non-limiting embodiment, the test strip of the present invention is comprised of four superimposed layers. The layers can be adhered to a base material through lamination with adhesives or through compression in a cassette with a top and bottom as depicted in FIG. 1, without the requirements of lamination.

Typically, the biological sample is whole blood taken from a subject's finger or heel-stick. In one non-limiting embodiment, the combination of layers in the test strip allows for zero percent bias in the range of 32 to 52% hematocrit and in the analytical range of 0.5 to 30 U/L (units per liter) total ADA and ADA2.

As shown in FIG. 1, in one non-limiting embodiment, the test strip comprises four layers, three of which are membranes which evenly spread the applied blood sample and separate blood components from plasma. The first layer is referred to as the sample spreading layer and is labeled as 1 in FIG. 1. The sample spreading layer distributes or meters the cells in the biological sample evenly across the surface of the primary membrane. The sample spreading layer provides a uniform concentration of cells between the interface of the spreading layer and the underlying primary membrane. The spreading layer can be a mesh material, an isotropically porous membrane (same porosity throughout), or an anisotropic membrane (a gradient in porosity). The spreading layer can be composed of nylon or polyester with a pore size in the range of 10-300 μm. Precise permeability of the spreading layer is critical, as it determines whether a homogeneous biological sample will be uniformly distributed across the surface of the underlying primary membrane layer. The surface of the spreading layer is in direct contact with the primary membrane for uniform transfer of the biological material through a lateral and vertical migration of the biological fluid.

The test strip further comprises a primary membrane layer labeled 2 in FIG. 1. Fluid of the biological sample flows transverse across the spreading layer 1 before migrating vertically into the primary membrane 2. The primary membrane layer is a blood separation membrane.

This primary whole blood separation membrane is also referred to herein as Membrane-1. In one non-limiting embodiment, Membrane-1 comprises a non-hemolytic surfactant, a hemagglutinating agent, a hemoglobin oxidizing agent that oxidizes hemoglobin to methemoglobin, a polymer, and buffer. Membrane-1 can be composed of one, or a combination of several, material(s) including, but not limited to, glass fiber, nylon, polyester, cellulose, cellulose acetate, nitrocellulose, polycarbonate, polyvinylidene difluoride, polyethersulfone, or polysulfone with a particle retention in the range of 2.0-10.0 μm. Membrane-1 is comprised of hemagglutinating agents, including but not limited to, anti-red blood cell antibodies, chitosan, hexadimethrine bromide, poly-L-lysine, poly-L-lysine hydrobromide, poly-D-lysine, poly-D-lysine hydrobromide, poly-DL-lysine hydrobromide, poly-L-arginine hydrochloride, poly(allylamine hydrochloride), poly(ethylenimine hydrochloride), diethylaminomethyl dextran, poly(n,n-dimethyl-3,5-dimethylene piperidinium chloride), or crude or purified lectins which agglutinate human type O erythrocytes efficiently such as those from Phaseolus vulgaris, Maclura pomifera, Ulex europaeus, and Solanum tuberosum. Additionally, the hemagglutinating agents can also be combined with a Neuraminidase, such as those from Clostridium perfringens, Arthrobacter ureafaciens, or Streptococcus pneumonia, to increase the hemagglutination efficiency of any lectins added to the primary blood separation membrane. The hemagglutinating agents can be immobilized together with a polymer, including but not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride). Non-limiting examples of hemoglobin oxidizing agents include potassium nitrite, sodium nitrite, potassium nitrate, sodium nitrate, potassium iodate and sodium iodate.

In one non-limiting embodiment, Membrane-1 contains hemolytic surfactants to liberate intracellular ADA1.

In this non-limiting embodiment, the test strip further comprises secondary membranes labeled 3A/3B in FIG. 1. The plasma and remaining cells from the primary membrane continue migrating vertically downward into the secondary membranes 3A/3B, also referred to herein as Membrane-2. Membrane-2 is in direct contact with Membrane-1. Membrane-2 is composed of one, or a combination of several, material(s) including, but not limited to, glass fiber, nylon, polyester, cellulose, cellulose acetate, nitrocellulose, polycarbonate, polyvinylidene difluoride, polyethersulfone or polysulfone with a pore size in the range of 0.8-5.0 μm. Membrane-2 comprises a non-hemolytic surfactant, polymer, and buffer.

Secondary membrane 3A further comprises erythro-9-(2-hydroxy-3-nonyl) adenine (EHNA), a potent inhibitor of adenosine deaminase-1 (ADA1). Immobilized EHNA ensures no interference by ADA1 on the serial colorimetric enzymatic redox mechanism, enabling specific determination of ADA2 levels.

Secondary membrane 3B does not contain EHNA and enables determination of total ADA activity.

As will be understood by the skilled artisan upon reading this disclosure, an alternative embodiment of this invention may include a test strip comprising a secondary membrane 3A for detection of ADA2 only and/or a separate test strip comprising a secondary membrane 3B for detection of total ADA only.

The optimal pH of ADA can range from about 6.0 to 9.0 depending on the variant. In one non-limiting embodiment, Membrane-2 contains an immobilized preconditioning buffer in the pH range of 6.0 to 9.0. By preconditioning the solution below the optimal pH of ADA, the kinetics of the enzymatic reaction is slowed which expands the analytical range. In addition, preconditioning of the biological fluid allows time for the homogenous mixing of the excipients while also buffering the biological fluid to a suitable pH for the enzymatic determination of ADA.

In one non-limiting embodiment, the components on Membrane-2 are immobilized with a polymer. Examples of polymers include, but are not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride).

In this non-limiting embodiment, the test strip further comprises a tertiary membrane labeled 4A/4B in FIG. 1. The tertiary membrane is also referred to herein as “the reagent membrane” or “Membrane-3”. The buffered fluid containing ADAs travels from Membrane-2 to the underlying Membrane-3. The reagent membrane is visually clean and smooth with submicron-sized pores thus providing excellent optical and reflective properties. Membrane-3 is composed of one, or a combination of several, material(s) including, but not limited to, nylon, cellulose, cellulose acetate, nitrocellulose, polycarbonate, polyethersulfone or polysulfone with a pore size in the range of 0.03-1.2 μm. This reagent membrane provides a uniform end-color in the read zone for precise detection. In one non-limiting embodiment, the reagent membrane is treated with a buffer, surfactant, stabilizers, an indicator, a cofactor, substrates, and/or enzymes, all of which are immobilized on the reagent membrane using a polymer. In one non-limiting embodiment, the reagent membrane for total ADA and/or ADA2 detection comprises the substrate adenosine, inorganic phosphate, enzymes purine nucleoside phosphorylase (PNP), xanthine oxidase (XTO), and peroxidase (electron mediator), and an indicator specific for oxidation. In one non-limiting embodiment, the indicator is a colorimetric indicator such as a monodye or Trinder Reagent that produces a colored product upon oxidation, with the product of oxidation having a lambda max wavelength between 500 nm and 700 nm.

In one non-limiting embodiment, the reagent membrane is buffered to a pH of 6.0-9.0. In one non-limiting embodiment, the components on Membrane-3 are immobilized with a polymer including, but not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride).

The biological fluid slowly migrates vertically downward onto the reagent membrane and total ADA and/or ADA2 levels in the sample are measured electrochemically or via end-color intensity and calibrated against a laboratory reference instrument.

More specifically, adenosine is converted to inosine through hydrolytic deamination of the amino group at the C6 position of adenosine. This conversion is catalyzed by adenosine deaminase enzymes. Hypoxanthine is derived from inosine by the enzyme nucleoside phosphorylase.

Hypoxanthine is then converted to uric acid and hydrogen peroxide (H₂O₂) by xanthine oxidase (XTO). In the presence of peroxidase and hydrogen peroxide, the indicator or indicators are oxidized to yield a colored product. Since the secondary membrane 3A comprises the ADA1 inhibitor EHNA, any conversion of adenosine measured there can be attributed to ADA2 while conversion of adenosine via second membrane 3B is attributable to total ADA in the biological sample.

In one non-limiting embodiment, end-color intensity of the reagent membrane is measured in percent reflectance units on a handheld meter or via an optical image measuring Red/Green/Blue (RGB) values and converted to U/L through a preprogrammed curve set. The concentration of U/L total ADA and ADA2 can be determined by the end-color intensity at a given time or by kinetic rate determination. In one non-limiting embodiment, the reagent membrane is positioned facing a light-emitting diode (LED) and photodiode to measure the end-color intensity of the reagent membrane or positioned facing a camera to image the end-color using RGB values. In one non-limiting embodiment, the LED and photodiode can detect the end-color of a generated from the tetrazolium salt to its oxidized formazan that has a lambda max wavelength in the range of 500 nm and 700 nm for reflectance determination.

In one non-limiting embodiment, calculated U/L ADA1 activity is determined by a device or mobile application by calculating the difference between measured total ADA and ADA2 activity.

The test strips, devices, kits, and methods of this invention can be used for the initial diagnosis, monitoring, and/or management of disorders associated with abnormal levels of ADA1 and/or ADA2.

Elevated ADA is an important biomarker with diagnostic utility in tuberculosis (Lamsal et al.

Southeast Asian J Trop Med Public Health 2007 38(2):363-9), celiac disease (Cakal et al. Journal of Clinical Laboratory Analysis 2010 24:323-326), juvenile rheumatoid arthritis and systemic lupus erythematosus (Hitoglou et al. Clinical Rheumatology 2014 20:411-416; Saghiri et al. Rheumatoid Int 2012 32(6):1633-8), and diabetes mellitus (Hoshino et al. Diabetes Res Clin Pract 1994 25(2):97-102).

Non-limiting examples of disorders associated with ADA deficiencies include, but are not limited to: 1) inflammation/vasculitis including cutaneous manifestations and stroke; 2) immune dysregulation including hypergammaglobulinemia, absent or low class-switch memory B cells, and inadequate response to vaccination; and 3) hematologic findings including pure red blood cell aplasia, immune mediated neutropenia, and pancytopenia. Furthermore, it is recognized that DADA2 is a relatively newly defined rare disease with a heterogeneous and complex presentation. Therefore, it can be expected that additional signs, symptoms, and manifestation will be associated with reduced or absent levels of ADA2, and these patients too will benefit from ADA2 testing as disclosed herein.

A current spectrum of clinical manifestations of reduced or absent ADA2 is summarized in Barron et al. (Frontiers in Immunology, 2022 12:811773). It is expected that the test strips, devices, kits, and methods disclosed herein will be useful in all of these clinical manifestations.

In its simplest form, the devices of this disclosure comprise a portable meter capable of reading electrochemical or colorimetric signals from a test strip and a unique test strip as disclosed herein for detection of ADA2 and/or total ADA.

The kits, referred to herein comprises the device with test strips, as well as means for obtaining, preparing, storing, and/or transporting the biological sample. Kits may also include directions of use for obtaining the sample and measuring ADA activity.

In one non-limiting example, the sample is transferred via fixed-volume capillary pipette.

In one non-limiting embodiment, the biological sample is pre-mixed with a diluent to lyse cells and liberate intracellular ADA1.

In one non-limiting embodiment, the results are transmitted to a computer or mobile application for data transformation, display, and storage.

In one non-limiting embodiment, the device is used to identify subjects with reduced ADA2 levels in need of prompt treatment with TNF inhibitors such as etanercept, adalimumab, infliximab, or golimumab. Examples of such subjects include, but are not limited to, pediatric patients presenting with stroke.

In another non-limiting embodiment, the device is used to identify subjects with elevated total ADA, ADA1, and/or ADA2 as a diagnostic biomarker in tuberculosis (Lamsal et al. Southeast Asian J Trop Med Public Health 2007 38(2):363-9), celiac disease (Cakal et al. Journal of Clinical Laboratory Analysis 2010 24:323-326), juvenile rheumatoid arthritis and systemic lupus erythematosus (Hitoglou et al. Clinical Rheumatology 2014 20:411-416; Saghiri et al. Rheumatoid Int 2012 32(6):1633-8), and diabetes mellitus (Hoshino et al. Diabetes Res Clin Pract 1994 25(2):97-102).

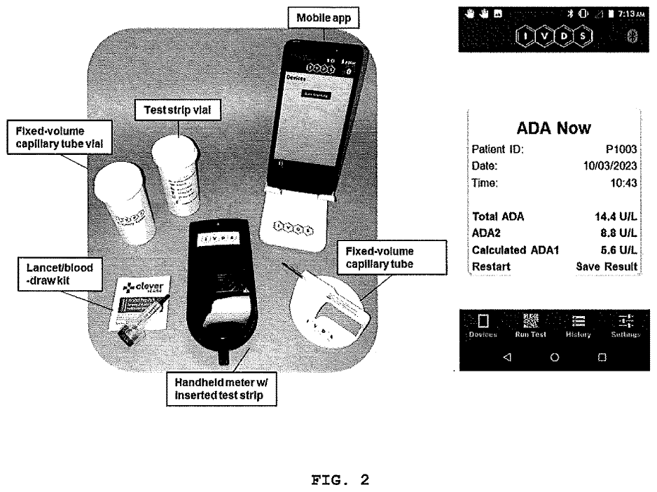

When the test strips, device, kit and methods herein are paired together as in the non-limiting embodiment depicted in FIG. 2, this invention provides a POCT for total ADA, ADA2, and calculated ADA1 which is much faster and less expensive compared to current standards and can be used in the clinic or for home monitoring without any requirements for a skilled technician or transport to a remote laboratory for testing.

As ADA1 and ADA2 deficiencies are relatively rare conditions, this POCT is expected to be very helpful in diagnosing as well as shortening the diagnostic journey experience for most patients with these disorders; this is especially true for DADA2 patients.

In addition, as elevated total ADA, ADA1 and ADA2 have been linked to tuberculosis (Lamsal et al. Southeast Asian J Trop Med Public Health 2007 38(2):363-9), celiac disease (Cakal et al. Journal of Clinical Laboratory Analysis 2010 24:323-326), juvenile rheumatoid arthritis and systemic lupus erythematosus (Hitoglou et al. Clinical Rheumatology 2014 20:411-416; Saghiri et al. Rheumatoid Int 2012 32(6):1633-8), and diabetes mellitus (Hoshino et al. Diabetes Res Clin Pract 1994 25(2):97-102), this POCT is also expected to be a useful diagnostic and/or monitoring tool for these conditions.

The following non-limiting example is provided to further illustrate the present invention.

### EXAMPLE

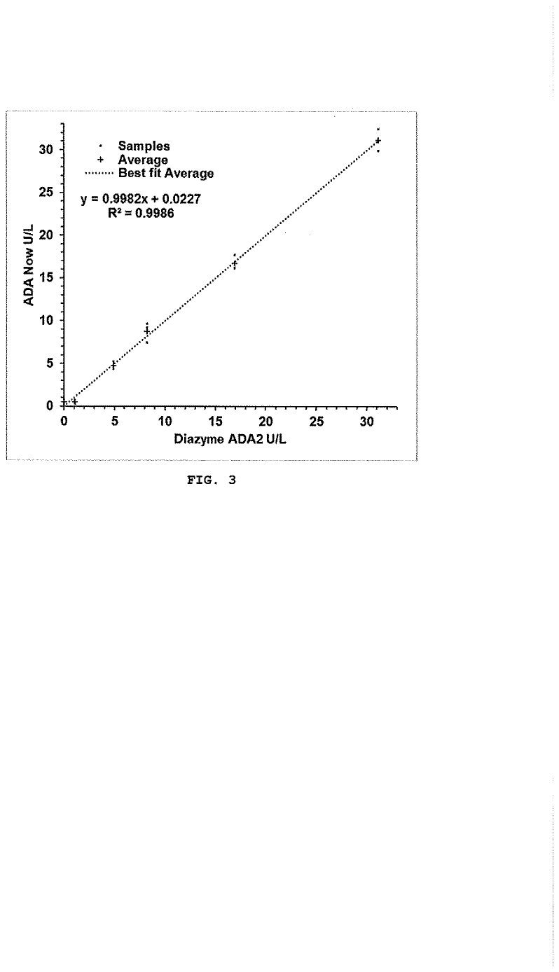

Samples of ADA2 deficient plasma were spiked with recombinant ADA2 to six levels. They were measured using an embodiment of the invention described herein (ADA Now) and simultaneously using a commercially available reference assay from Diazyme Laboratories Inc. (Poway, CA). The calculated ADA2 activity from ADA Now was plotted against the results from the reference to achieve a linear agreement. Results are depicted in FIG. 3.

1: A device for quantitatively measuring adenosine deaminase 2 (ADA2) in a biological sample, said device including a test strip comprising:
a sample spreading layer;
a primary whole blood separation membrane;
a secondary whole blood separation membrane comprising erythro-9-(2-hydroxy-3-nonyl)adenine; and
a reagent membrane for detection of ADA2 activity comprising adenosine, inorganic phosphate, purine nucleoside phosphorylase, xanthine oxidase, peroxidase and an indicator.

2: A device for quantitatively measuring total adenosine deaminase (ADA) in a biological sample, said device including a test strip comprising:
a sample spreading layer;
a primary whole blood separation membrane;
a secondary whole blood separation membrane; and
a reagent membrane for detection of total ADA activity comprising adenosine, inorganic phosphate, purine nucleoside phosphorylase, xanthine oxidase, peroxidase and an indicator.

3: A device for quantitatively measuring total adenosine deaminase (ADA) and adenosine deaminase 2 (ADA2) in a biological sample, said device including a test strip comprising:
a sample spreading layer;
a primary whole blood separation membrane;
a sectioned secondary whole blood separation membrane comprising regions with or without erythro-9-(2-hydroxy-3-nonyl) adenine; and
a reagent membrane for detection of ADA2 activity and total ADA activity comprising adenosine, inorganic phosphate, purine nucleoside phosphorylase, xanthine oxidase, peroxidase and an indicator.

4-15: (canceled)

16: The device of claim 1 wherein the test strip allows for zero to six percent bias in the hematocrit range of 32-52% through the analytical range of 0.5-30 U/L of the ADA.

17: A method for quantitatively measuring ADA2 in a biological sample, said method comprising applying the biological sample to the device of claim 1 and measuring the ADA2 in the biological sample.

18: The method of claim 17 wherein the biological sample applied is less than 25 μL.

19-21: (canceled)

22: The method of claim 17 used in the diagnosis of a deficiency in ADA1 or ADA2 or elevated ADA1 or ADA2.

23: (canceled)

24: A kit for detecting total ADA and/or ADA2 in a biological sample and/or calculating ADA1 activity in the biological sample, said kit comprising the device of claim 1, a means for obtaining a blood sample for testing and a portable hand-held meter for reading test strips of the device.

25: The kit of claim 24 further comprising a mobile device application for quantitative analysis and/or transmitting data from the test strip to a health care provider.

26: The device of claim 2 wherein the test strip allows for zero to six percent bias in the hematocrit range of 32-52% through the analytical range of 0.5-30 U/L of the ADA.

27: The device of claim 3 wherein the test strip allows for zero to six percent bias in the hematocrit range of 32-52% through the analytical range of 0.5-30 U/L of the ADA.

28: A method for quantitatively measuring total ADA in a biological sample, said method comprising applying the biological sample to the device of claim 2 and measuring the total ADA in the biological sample.

29: The method of claim 28 wherein the biological sample applied is less than 25 μL.

30: The method of claim 28 used in the diagnosis of a deficiency in ADA1 or ADA2 or elevated ADA1 or ADA2.

31: A method for quantitatively measuring ADA2 and total ADA in a biological sample, said method comprising applying the biological sample to the device of claim 3 and measuring the ADA2 and total ADA in the biological sample.

32: The method of claim 31, further comprising calculating ADA1 activity from the difference between measured total ADA and ADA2 activity.

33: The method of claim 31 wherein the biological sample applied is less than 25 μL.

34: The method of claim 31 used in the diagnosis of a deficiency in ADA1 or ADA2 or elevated ADA1 or ADA2.

35: A kit for detecting total ADA and/or ADA2 in a biological sample and/or calculating ADA1 activity in the biological sample, said kit comprising the device of claim 2, a means for obtaining a blood sample for testing and a portable hand-held meter for reading test strips of the device.

36: A kit for detecting total ADA and/or ADA2 in a biological sample and/or calculating ADA1 activity in the biological sample, said kit comprising the device of claim 3, a means for obtaining a blood sample for testing and a portable hand-held meter for reading test strips of the device.
